(19) United States
(12) Patent Application Publication
(10) Pub. No.: US 2025/0000390 A1
(43) Pub. Date: Jan. 2, 2025

(54) MULTIMODAL METHOD AND SYSTEM FOR EVALUATING AND IMPROVING NEUROMOTOR CONTROL ABILITY

(71) Applicant: PRISM NEURO PTY LTD, Braddon (AU)

(72) Inventor: Elizabeth McGrath, Griffith (AU)

(73) Assignee: PRISM NEURO PTY LTD, Braddon (AU)

(21) Appl. No.: 18/697,796

(22) PCT Filed: Oct. 5, 2022
(86) PCT No.: PCT/AU2022/051188

§ 371 Date: Apr. 2, 2024

(30) Foreign Application Priority Data
Oct. 7, 2021 (AU) 2021903207

(51) Int. Cl. A61B 5/11; A61B 3/028; A61B 3/11; A61B 5/00

(52) U.S. Cl. CPC A61B 5/1124; A61B 3/028; A61B 3/112; A61B 5/4023; A61B 5/6803; A61B 5/702; A61B 2503/10; A61B 2505/09

(57) ABSTRACT

A method and system for evaluating an individual's neuromotor control ability through conducting a multimodal series of tests, said method comprising the steps of capturing the data from each test, assessing the test data to generate a result for each test, comparing the results of the tests to normative standards, and combining the results of the multiple tests into a single overall score, said multimodal series of tests comprising functional tests of the three main movement control sensory systems being the proprioceptive, visual and vestibular systems.

### FIELD OF THE INVENTION

The present invention relates generally to a multimodal method and system for evaluating and improving neuromotor control ability. In particular, the method and the system defined in the present invention can be used for evaluation and testing of individuals for assessing their athletic potential and risk of injury, as an aid to diagnosing conditions that affect neuromotor control, and for monitoring recovery from conditions that affect neuromotor control. The method and system of the present invention can also be used in rehabilitation and training for injury prevention and improving neuromotor control.

### BACKGROUND OF THE INVENTION

The evolution of human movement required the development of the ability to dynamically stabilise the body's weight through interactions of the body's trunk and limbs with both moving and stationary objects. This ability includes stabilising the entire bodyweight through a single ankle and foot during walking or running activity. Stability during movement is maintained by a complex interaction between bony anatomy, ligamentous integrity and neuromotor control that consists of sensory reception, central nervous system (CNS) integration of sensory information, and neural signals for muscular action.

Neuromotor control is a result of the action of the sensorimotor system. The sensorimotor system consists of the interaction between the peripheral sensory receptors, central nervous system processing and neural-muscular output. It can be most specifically defined as the system of sensory, motor and central integration and processing components involved with maintaining joint homeostasis during functional activities. The primary sensory input required for joint homeostasis is received from the visual, vestibular and proprioceptive systems. These senses provide information to the brain about the environment and the body's relative position and interaction with the environment. The brain integrates and processes this information in a continual feedback loop to calculate and deliver motor signals to activate relevant muscles in order to maintain dynamic stability.

Prior art methods for the assessment of neuromotor control ability are primarily focused on test methods that measure the quality of movement i.e., how well an individual can execute a given movement task, and not the function of the underlying control mechanisms that enable the movement. The inventor of the present invention has found that an assessment of the movement does not provide the insight necessary for understanding poor movement, or for improving movement. This comes from an assessment of the components that underpin neuromotor control ability. Studies show that poor performance on some of these components increase risk of injury, while excellent scores on some components are an indicator of athletic potential. In addition, neuromotor control ability is affected by injury to either the brain or musculoskeletal system, by certain diseases, and by ageing. A measure of neuromotor control ability can also be indicative of these conditions.

### SUMMARY OF THE INVENTION

This invention relates to a method and system to test, rate and improve an individual's neuromotor control ability. Neuromotor control relies on three main sensory systems to provide continuous feedback for neural processing by the brain, in order for it to send and adjust motor signals to the body's muscles. These sensory systems are the proprioceptive, visual, and vestibular systems, and the current invention tests the function of these three pathways. In accordance with the present invention, the output of the testing is combined with some information about the individual and the result is used to rate the individual's neuromotor control ability. If the score meets a certain predetermined standard, the individual will have athletic potential, if the score is less than the standard, the individual is at risk of injury, or is suffering the effects of injury or disease, and should do particular exercises, or rest and recover, to minimise this risk.

The essence of the present invention is a method for evaluating an individual's neuromotor control ability. This is achieved through conducting a multimodal series of tests, capturing the data from each test, assessing the test data to generate a result for each test, comparing the results of the tests to normative standards, and combining the results of the multiple tests into a single overall score. The single score can be used as a convenient screening tool for groups of people, such as a sports team, and can also be used for evaluating an individual. The testing may be repeated at intervals allowing the score to be tracked over time for comparison to baseline testing and can be deconstructed to provide insight into individual performance when their score or score trend is of concern.

The multimodal test method comprises functional tests of the three main sensory systems, proprioception, visual and vestibular, that provide the feedback control mechanism for human movement. This method includes, at a minimum, a digitally enabled test for proprioceptive acuity, combined with at least one of the following digitally-enabled tests: an eye tracking test to assess the vestibular and visual systems, a pupillometry test to assess the visual system, simultaneous eye tracking and pupillometry testing, or a test incorporating unstable platform balance to assess the vestibular system.

The tests can also be performed repetitively, without capturing data for assessment, as a means of providing training to improve the function of the sensory pathways, thereby improving an individual's neuromotor control ability.

### DETAILED DESCRIPTION OF THE INVENTION

One of the components of the multimodal test method is a digitally enabled test for proprioceptive acuity. Proprioception is the body's sensory system for joint position and force. This enables the brain to know the position of the body. As an example, a test using a novel apparatus for the individual's ability to distinguish between small movement increments at a body joint is used. In one example, the ankle joint is tested. The individual places one foot along the axis of a rotatable plate that is parallel to the floor and is held in a box a small distance above the floor. The individual can use the bottom of their foot, by making a rotational movement of their ankle joint, to rotate one side of the plate down a small amount from horizontal until the plate comes to a stop. The box is configured to stop the plate's rotation in one of a number of predetermined positions below the horizontal. The individual can then rotate the plate back to the horizontal by making a corresponding rotation of their ankle until the bottom of their foot is horizontal. The stop position is reset. The individual can then rotate the plate down a second time to the new stop position that may or may not be the same as the first stop position. The individual is instructed to report whether the second stop position is the same as, lower than, or higher than, the first stop position. The test consists of a number of pairs of plate stop positions that the individual must compare and report.

In another version of the test the individual is first given a practice session where they have the opportunity to feel each of the stopping positions prior to beginning the test. The stopping positions are numbered so that the individual is able to associate a numbered position with an amount of rotation of their ankle joint. During the test, the stopping positions are presented randomly to the individual and the individual must report which stop position number, and hence which ankle rotation position, they feel each time they rotate the plate. In both versions of the test the plate stop positions are controlled by a computer program and the individual's responses are input to, and recorded by, a computer program. At the completion of the test the responses are assessed using a computerised algorithm and a result is generated.

The next component of the exemplary multimodal test method is the eye tracking test to assess the vestibular and visual systems. The vestibular system is the body's sensor system for detecting movement, acceleration and the body's position relative to gravity, and this information has a critical role is enabling movement. In addition, information from the vestibular system is necessary for stabilising eye movements. The vestibular system must accurately assess whether the body is still or moving and in what direction, and how fast it is moving. This information is directly used to generate compensatory eye movements for the detected movement. This allows continuous optimised visual input during movement of the body. Similarly, when the body is still, this must be accurately reflected. An inability to perform eye movements, or performing them poorly, can therefore indicate a performance issue with the vestibular system. In addition, if eye movements are not optimised, the ability to focus on a target is reduced with a resulting decrement in visual system performance. Conversely, research has shown superior performance in eye movement ability correlates with better athletic performance.

The eye tracking testing component for the visual and vestibular systems relies on infrared digital videography that can capture images of an individual's eyes over time, process the images to determine gaze point location, and record the output as digital data files. This capability is provided by high-end virtual reality (VR) goggles in combination with a laptop computer. These VR goggles incorporate infrared light projectors and infrared video cameras that capture eye images from the infrared light reflected from the individual's eyes. The VR goggle software uses artificial intelligence algorithms to find the pupil edge and center in the eye image, and then calculates gaze point from the image data. This data is then available as an output stream for each eye for further processing. This capability could also be provided by any another arrangement for conducting infrared digital videography, or visible spectrum digital videography, of the eyes while they are looking at a display, with software visual stimulus presented to the individual by means of the display, with digital image processing software to provide gaze point position over time as digital data files.

The invention uses visual stimulus patterns in software that are uploaded to a VR headset. The test method requires the individual to look at the visual stimulus in the headset display. The individual's pupil gaze point position is downloaded from the VR goggles and analysed using appropriate algorithms. Tests include smooth pursuit (how accurately a participant can track a smoothly moving target), saccadic pursuit (how quickly/accurately a participant can locate a rapidly appearing target) and gaze stabilization (how well a participant can maintain focus on a target).

As an example, the individual's ability to track a smoothly moving target is assessed. The individual is instructed to look at the VR goggle display where a stimulus is presented. A computer program presents the stimulus to the display. Digital video cameras are focused on the individual's eyes and record the eyes as they move while looking at the stimulus. During the test the individual is instructed to follow a smoothly moving target, the stimulus, with their eyes. The digital video signal is captured by a computer program and computerised algorithms use the video data to calculate the position of each eye. Another algorithm analyses the eye position data to see how well each eye performed in the task, and whether the two eyes have any differences in their performance, to calculate a result.

In another example the individual's ability to maintain focus on a target is assessed. In another example the individual's ability to shift their focus from target to target is assessed.

The next component of the exemplary multimodal test method is the pupillometry test to assess the visual system performance. The following description refers to the use of VR goggles for this component test. As with eye tracking, this could also be accomplished with any other arrangement for infrared or visible spectrum digital videography of the eyes, with digital image processing of the eye images. In the case of pupillometry the images are processed to produce pupil diameter over time for each eye, and this data is provided as digital data files.

A key function of the eye is to regulate the amount of light that enters the eye and strikes the retina. The ability to contract and dilate the pupil is the mechanism for this light regulation. The pupillometry testing component of the invention involves stimulating an individual's eyes with a software program that provides novel light stimulus patterns that are uploaded to, and presented to the individual in, the VR goggle display. On-axis and off-axis light stimuli are presented to both eyes, left eye only and right eye only. The individual's pupil size vs time data is downloaded from the VR headset. Novel software algorithms analyse this data to calculate pupillary parameters including constriction and dilation velocities and machine learning algorithms may be applied to the entire test sequence to calculate a result for each eye. The results provide insights into the functioning of the visual system and sympathetic and parasympathetic branches of the autonomic nervous system. The method uses both static pupillometry where the eyes are still, and a novel embodiment of dynamic pupillometry testing. In dynamic pupillometry the individual is instructed to move their eyes to look at the light stimuli.

In a novel embodiment of the visual vestibular systems testing component, simultaneous pupillometry and gaze tracking is used. A software programmed visual stimulus pattern is uploaded to the VR goggles. The user is instructed to look at features of the visual stimulus in different locations, requiring eye movement. The stimulus includes on-axis and off-axis illumination that is presented to each of: both eyes, left eye only and right eye only. Data streams for both of: eye position vs time, and pupil diameter vs time, are captured simultaneously for both eyes. This novel method of simultaneous stimulation and data recording for both eye tracking and pupillometry testing is superior to the current art because it provides a more efficient test method over doing these tests separately. In addition, pupillometry is traditionally carried out with eyes stationary. Analysing pupil response during eye movement with complex stimulus patterns, provides increased testing sensitivity and insights not available through the current art, as does the simultaneous combination of eye movement characteristics with pupil response characteristics.

The next component of the exemplary multimodal test method is the test incorporating unstable platform balance to assess the vestibular system. In this component test an individual stands on a curved board that is fitted with an accelerometer. The individual attempts to keep the board level and still for 60 seconds. The individual next repeats this test with their eyes closed. During each 60 second component the output of the accelerometer vs time is captured in a digital data file. The data is analysed to determine accelerations, velocities, and displacements of the board during the testing periods. The amount of degradation in performance, i.e.—increased acceleration, velocity and displacement of the board, from the eyes open to the eyes closed condition, indicates level of vestibular system function.

The overall result of the exemplary multimodal test method is determined by considering each of the individual's component results. Each component result is compared with normative results for the component test. The individual's component result is given a rating based on how it compares with the normative results. The comparison and rating of individual component results is accomplished through the use of computer algorithms. This is repeated for each component test. The ratings for all components are then processed, using a computer algorithm, to achieve an overall rating of the movement control system. This overall rating is useful for managing groups of individuals, such as a sports team. The single score can be calculated and reviewed on a regular basis allowing the team manager to have a useful piece of information for decision making about the team. The score trends for individuals over time can be monitored to alert the team manager to potential issues. An individual's score can then be deconstructed to allow for investigation of the problem.

Scores for individuals not associated with a group can also be used as a diagnostic aid or to monitor conditions over time.

### DETAILED DESCRIPTION OF THE INVENTION WITH REFERENCE TO DRAWINGS

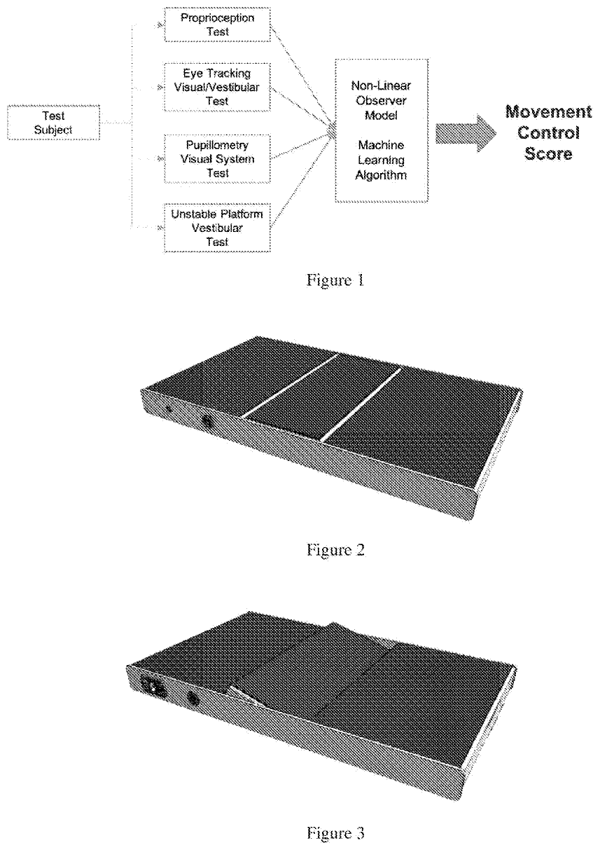

FIG. 1 is schematic illustration of the multimodal test method.

### Proprioceptive Acuity

The digitally enabled test for proprioceptive acuity is conducted using a box-like apparatus that has a rotatable plate that is parallel to the ground. The individual places one foot on the rotatable plate. The individual moves the rotatable plate with their foot. The box-like apparatus is equipped with an electronically controlled mechanism to stop the plate in one of five positions. This apparatus is automated using linear solenoids that push stop pins out at different heights under the lateral edges of the plate to form the plate stopping mechanism.

FIG. 2 shows the box-like apparatus for testing proprioceptive acuity in accordance with an embodiment of the invention. The box-like apparatus is designed to allow an individual with bare feet in standing position to place one foot on the rotatable plate that has an axis of rotation positioned centrally. This axis allows the plate to be tipped by the individual using one foot from a flat starting position, into a tilted position. The range of rotation of the plate is determined by a set of deployable stop pins (stopping mechanism), that can be extended from the side of the box-like apparatus under the lateral edges of the rotatable plate. An equivalent set of stop pins, or stopping mechanism, is present in each side of the box-like apparatus to deploy pins under each lateral edge of the rotatable plate. The height of a deployed stop pin determines the depth to which the rotatable plate can drop, and hence the final angle of rotation of the plate. The pin heights are set to allow rotations from 0 degrees (plate horizontal) to 15 degrees depending on the desired test pattern. The surfaces of the deck of the box-like apparatus are covered with a textured rubber to provide texture for sensory stimulation through the soles of the feet and good grip to ensure safety. The stop point of the rotatable plate is raised and lowered by means of a computer program operating on a control unit, that sends signals, by either wired or wireless means, to a circuit that is enclosed in the box-like apparatus. Based on these signals, the circuit energises the desired solenoid which in turn deploys the associated position pin forming a stopping mechanism for the rotation of the plate. This enables the rotatable plate to be stopped in pre-set increments, to give fixed changes in the angle of tilt of the footplate.

FIG. 3 is an image of the box-like apparatus showing the rotatable plate in a tilted position.

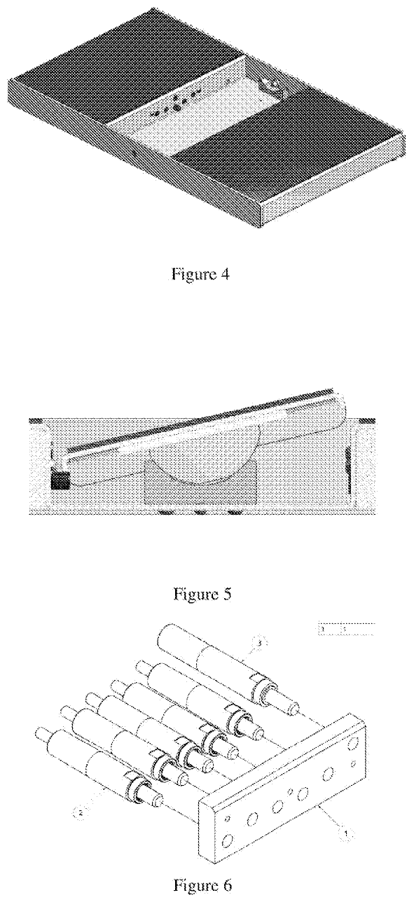

FIG. 4 shows the box-like apparatus with the rotatable plate removed to provide a view of the deployable stop pins on one side of the apparatus. The same arrangement is present on the opposite side of the apparatus.

FIG. 5 shows a cross section of the rotatable plate in a tilted position determined by a deployed stop pin.

FIG. 6 shows the stopping mechanism assembly consisting of the pin position mounting block, five push-type solenoid assemblies, one pull-type solenoid assembly.

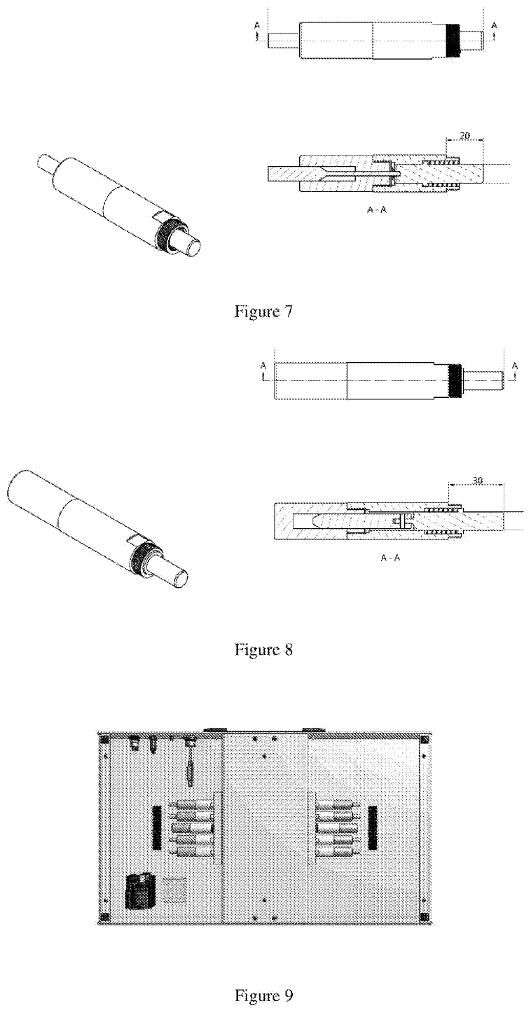

FIG. 7 shows the detail of a push-type solenoid assembly. These assemblies utilise cylindrical body solenoids which have a threaded end allowing them to be directly screwed into the solenoid mounting bodies. It should be noted that a similar arrangement could be achieved with box-body push-type solenoids which would be attached to the solenoid mounting body through the use of L-brackets attached to both the solenoid and the solenoid mounting body. In this case, the shape of the solenoid mounting body would need to be altered to accommodate the L-bracket attachment. It is also possible that the return spring could be relocated to the solenoid plunger instead of the position pin, allowing direct mounting of the box-body solenoid, through the L-bracket attachment, to the pin position mounting block, removing the need for solenoid mounting bodies.

FIG. 8 shows the detail of a pull-type solenoid assembly. These assemblies utilise cylindrical body solenoids which have a threaded end allowing them to be directly screwed into the solenoid mounting bodies. It should be noted that a similar arrangement could be achieved with box-body pull-type solenoids which would be attached to the solenoid mounting body through the use of L-brackets attached to both the solenoid and the solenoid mounting body. In this case, the shape of the solenoid mounting body would need to be altered to accommodate the L-bracket attachment.

FIG. 9 shows a bottom view of the box-like apparatus. The stopping mechanisms for each side of the rotatable plate can be seen.

While various embodiments of the present invention have been described above, it should be understood that they have been presented by way of example only, and not by way of limitation. It will be apparent to a person skilled in the relevant art that various changes in form and details can be made therein to suit different situations without departing from the spirit and scope of the present invention. Thus, the present invention should not be limited by any of the above-described exemplary embodiments.

1. A method for evaluating an individual's neuromotor control ability through conducting a multimodal series of tests, said method comprising the steps of capturing the data from each test, assessing the test data to generate a result for each test, comparing the results of the tests to normative standards, and combining the results of the multiple tests into a single overall score, said multimodal series of tests comprising functional tests of the three main movement control sensory systems being the proprioceptive, visual and vestibular systems wherein the said method further comprising the steps of:
a. using one or more digitally enabled test for proprioceptive acuity, a digitally enabled eye tracking test to assess the vestibular and visual systems, a digitally enabled pupillometry test based on dynamic pupillometry undertaken while the subject's eyes are moving to look at complex stimulus patterns to assess the visual system, and a test incorporating unstable platform balance to assess the vestibular system;
b. placing one foot of an individual along the axis of a rotatable plate that is parallel to the floor and held in a box, a small distance above the floor and configured to stop in one of a number of predetermined positions below the horizontal, the said individual using the movement of individual's ankle joint to rotate the said plate a small amount from horizontal until the plate comes to a stop and the individual rotating the plate back to the horizontal and then rotating the plate down a second time to a stop position that may or may not be the same as the first stop position and the individual being instructed to report whether the second stop is the same as, lower than, or higher than, the first stop;
c. presenting to the individual random stopping positions and asking the individual to report which stopping position they feel each time they rotate the plate.
d. controlling the plate stopping positions by a computer program and recording the individual's responses by the said computer program assessing the responses using a computerised algorithm and generating a result at the completion of the test; and
e. carrying out the said visual and vestibular testing using simultaneous eye tracking and dynamic pupillometry.

2. The method of claim 1 wherein the vestibular component testing is carried out using a curved board with an accelerometer for conducting a balance test with eyes open and comparing it with a balance test with eyes closed.

3. A method for improving movement control ability through repetitive training for any of the three key sensory systems, proprioceptive, visual and vestibular, using the test methods of claim 1 on a scheduled repetitive basis without capturing and analysing data.

4. A system for evaluating an individual's movement control ability through conducting a multimodal series of tests, said system comprising means for testing the proprioceptive, visual and vestibular systems wherein the said system further includes:
a. a means for carrying out pupillometry, a means for carrying out eye tracking, a means for carrying out simultaneous eye tracking and pupillometry and an apparatus for testing proprioceptive acuity;
b. said apparatus for testing proprioception comprises a rotatable plate that is parallel to the floor and is held in a box, a small distance above the floor and configured to stop in one of a number of predetermined positions below the horizontal by use of a stopping mechanism assembly wherein an individual is able to rotate the said plate a small amount from horizontal until the plate comes to a stop using the movement of said individual's ankle joint wherein the said stopping mechanism assembly comprises one or more of push type solenoid assemblies and one or more of pull type solenoid assemblies or other linear actuators and configured to allow the individual to use the movement of the ankle joint to rotate the plate back to the horizontal and then rotating the plate down a second time to a stop at a position that may or may not be the same as the first stop position;
c. said means for visual and vestibular tests include an eye tracking testing component that uses infrared digital videography that can capture an individual's pupil size and position over time and record the output as digital data files;
d. a means for randomly presenting the said stopping positions to an individual wherein said plate stopping positions are controlled by a computer program which inputs and records individual's responses;
e. a computer program operating on a control unit, that sends signals, by either wired or wireless means, to a circuit that is enclosed in the box-like apparatus to raise or lower the stop point of the rotatable plate; and
f. software for conducting and analysing simultaneous eye tracking and dynamic pupillometry assessments with the use of a digital videography system such as VR goggles for testing visual and vestibular system performance.

5. A system as defined in claim 4 wherein the means for vestibular component testing includes a curved board with an accelerometer for conducting a balance test with eyes open and a means for comparing it with a balance test with eyes closed.

6. The system of claim 5 wherein the visual vestibular systems testing includes simultaneous pupillometry and gaze tracking using a software programmed visual stimulus pattern that is uploaded to the VR goggles.

7. The system of claim 6 wherein the stimulus of the software programmed visual stimulus pattern includes on-axis and off-axis illumination that is presented to each of both eyes, left eye only and right eye only.

8. The system of claim 7 wherein the time varying waveforms for pupil size and pupil position are analysed using software with artificial intelligence algorithms.

9. The system of claim 8 wherein the system is enabled using computer software embodied in a computer-readable medium.

10. A method for improving movement control ability through repetitive training for any of the three key sensory systems, proprioceptive, visual and vestibular, using the test methods of claim 2 on a scheduled repetitive basis without capturing and analysing data.
